(19) United States
(12) Patent Application Publication
(10) Pub. No.: US 2023/0371824 A1
(43) Pub. Date: Nov. 23, 2023

(54) ASSESSING A CARDIAC CONDITION INDEPENDENTLY OF PPG WAVEFORM AMPLITUDE ANALYSIS

(71) Applicant: THE JOHNS HOPKINS UNIVERSITY, Baltimore, MD (US)

(72) Inventor: Harry A. SILBER, Owings Mills, MD (US)

(73) Assignee: THE JOHNS HOPKINS UNIVERSITY, Baltimore, MD (US)

(21) Appl. No.: 18/248,722

(22) PCT Filed: Oct. 12, 2021
(86) PCT No.: PCT/US2021/054544

§ 371 Date: Apr. 12, 2023

Related U.S. Application Data
(60) Provisional application No. 63/092,403, filed on Oct. 15, 2020.

(51) Int. Cl. A61B 5/0205; A61B 5/024; G16H 50/20

(52) U.S. Cl. CPC A61B 5/0205; A61B 5/02416; G16H 50/20

(57) ABSTRACT

In one example aspect, a computer-implemented method includes receiving biometrics data for a user; determining, based on the biometrics data, a baseline root-mean square (RMS) value prior to performance of a medical maneuver by the user; determining a minimum RMS value of the biometrics data during the medical maneuver; calculating a ratio between the minimum RMS value and the baseline RMS value to generate a minimum RMS ratio; and storing or outputting the minimum RMS ratio.

### CROSS-REFERENCE TO RELATED APPLICATIONS

This application claims priority to U.S. Provisional Patent Application No. 63/092,403 filed on Oct. 15, 2020, the disclosure of which is incorporated herein by reference.

### BACKGROUND

A patient's fluid status, state of congestion, or left heart filling pressure (LHFP) may be assessed for a variety of clinical purposes, such as diagnosing the causes of dyspnea, assessing volume status in critically ill patients, managing fluid output in chronic heart failure, etc. The peripheral blood pressure pulse amplitude response to a Valsalva maneuver (an expiratory effort against a closed throat) reflects a patient's fluid status and LHFP.

A Valsalva maneuver may be performed by moderately forceful attempted exhalation against a closed airway, usually done by closing one's mouth, pinching one's nose shut while making an effort to expel air out as if blowing up a balloon. Variations of the maneuver can be used either in medical examination as a test of cardiac function and autonomic nervous control of the heart, or to clear the ears and sinuses (that is, to equalize pressure between them) when ambient pressure changes, as in diving, hyperbaric oxygen therapy, or air travel.

A photoplethysmogram (PPG) is an optically obtained plethysmogram that can be used to detect blood volume changes in the microvascular bed of tissue. A PPG is often obtained by using a pulse oximeter which illuminates the skin and measures changes in light absorption. A conventional pulse oximeter monitors the perfusion of blood to the dermis and subcutaneous tissue of the skin.

With each cardiac cycle the heart pumps blood to the periphery. The pressure pulse distends the arteries and arterioles in the peripheral subcutaneous tissue. The continuously varying photoplethysmography waveform reflects the continuously varying pressure and pulse volume at the site that the PPG probe is placed.

An arrhythmia is a problem with the rate or rhythm of the heartbeat. During an arrhythmia, the heart can beat too fast, too slowly, or with an irregular rhythm. One type of arrhythmia is atrial fibrillation, which is an irregular and often rapid heart rate that occurs when the two upper chambers of your heart experience chaotic electrical signals. The result is a fast and irregular heart rhythm. Another type of arrhythmia is when frequent atrial or ventricular premature beats are present.

When an irregular rhythm is present, it introduces variability and inaccuracy in the calculation of pulse amplitude. This is because pulse amplitude is determined by measuring the difference between the peaks and valleys of the cycles being analyzed. In an irregular rhythm, the peaks and valleys vary with each beat. It would be beneficial to determine the overall size of the waveform by taking into account every point in the cardiac cycle.

### SUMMARY

In one example aspect, a computer-implemented method includes: receiving biometrics data for a user; determining, based on the biometrics data, a baseline root-mean square (RMS) value prior to performance of a medical maneuver by the user; determining a minimum RMS value of the biometrics data during the medical maneuver; calculating a ratio between the minimum RMS value and the baseline RMS value to generate a minimum RMS ratio; and storing or outputting the minimum RMS ratio

In another example aspect, a computer program product includes a computer readable storage medium having program instructions embodied therewith. The program instructions are executable by a computing device to cause the computing device to perform operations including: receiving biometrics data for a user; determining, based on the biometrics data, a baseline root-mean square (RMS) value prior to performance of a medical maneuver by the user; determining a minimum RMS value of the biometrics data during the medical maneuver; calculating a ratio between the minimum RMS value and the baseline RMS value to generate a minimum RMS ratio; and storing or outputting the minimum RMS ratio.

In another example aspect, a system includes a processor, a computer readable memory, a non-transitory computer readable storage medium associated with a computing device, and program instructions executable by the computing device to cause the computing device to perform operations including: receiving biometrics data for a user; determining, based on the biometrics data, a baseline root-mean square (RMS) value prior to performance of a medical maneuver by the user; determining an RMS value of the biometrics data at the end of the medical maneuver; calculating a ratio between the RMS value at the end of the medical maneuver and the baseline RMS value to generate an RMS ratio; and storing or outputting the RMS ratio.

### BRIEF DESCRIPTION OF THE DRAWINGS

FIG. 1 illustrates an example overview and environment in accordance with aspects of the present disclosure.

FIG. 2 illustrates an example flowchart of an example process for determining a minimum RMS ratio that may used in a medical analysis.

FIG. 3 illustrates an example flowchart of an example subprocess for calculating a baseline RMS value.

FIG. 4 illustrates an example flowchart of an example subprocess for determining a minimum RMS value during performance of a medical maneuver.

FIG. 5 illustrates an example diagram for advancing time windows to determine a minimum RMS value during performance of a medical maneuver.

FIG. 6 illustrates example components of a device that may be used within environment of FIG. 1.

### DETAILED DESCRIPTION

A Valsalva maneuver may sometimes be used either for medical analysis (e.g., a medical examination, test, study, etc.) as a test of cardiac function. The change in amplitude of a finger photoplethysmography (PPG) waveform during a Valsalva maneuver reflects a patient's fluid status, state of congestion, or cardiac filling pressure. However, determining “peaks and valleys” (e.g., representing amplitude changes) in a waveform may not be clear or accurate in patients having arrhythmias such as atrial fibrillation, premature ventricular complexes, etc. For example, the waveform of a PPG in a patient having an arrhythmia may be erratic, making it difficult to accurately identify the amplitudes and amplitude changes in the waveform. Accordingly, aspects of the present disclosure may include a system and/or method that may analyze a medical condition (e.g., cardiac function) independently of analyzing waveform amplitude changes. For example, aspects of the present disclosure may determine a ratio between a minimum root-mean square value of a patient's PPG during performance of a Valsalva maneuver and a baseline RMS value of the PPG prior to performance of the Valsalva maneuver. This ratio may be used as part of a medical analysis, such as an assessment of fluid status, state of congestion, or cardiac filling pressure (e.g., left heart filling pressure (LHFP)), or other type of medical analysis/examination. That is, the techniques described herein do not rely on identifying waveform amplitudes, as these amplitudes may be inaccurate and/or difficult to calculate in patients with arrhythmias. In this way, a patient's cardiac filling pressure may be assessed even when that patient has an arrhythmic heartbeat. In some embodiments, the techniques described herein may be used for other types of medical assessments, in addition to, or instead of assessing cardiac filling pressure.

As described herein, aspects of the present disclosure may assess the change in a PPG waveform in way that is not dependent upon identifying peaks and valleys in the wave form. In some embodiments, aspects of the present disclosure may calculate the root-mean-square (RMS) of squares of individual PPG measurement points in over a period of time, which may provide a standard deviation of the waveform segment. A ratio between the minimum RMS during the Valsalva maneuver divided by the RMS of the baseline waveform, or the minimum RMS ratio, may be determined, and this the RMS ratio may provide information that may be used to assess cardiac filling pressure (or perform other types of medical analysis, such as fluid status or state of congestion). Additionally, or alternatively, the RMS near the end of a Valsalva maneuver divided by the RMS of the baseline waveform, or RMS ratio, may be determined. Aspects of the present disclosure may use of some or all the points within cardiac cycles, not just the peaks and valleys. As such, a richer set of data may be leveraged for performing a variety of medical analysis.

In accordance with aspects of the present disclosure, a user (e.g., patient, study participant, etc.) may perform a Valsalva maneuver, and a sensor system may obtain biometrics data from the user before and during the Valsalva maneuver from and provide this sensor data to an analysis system. For example, a PPG finger probe may measure changes in light absorption in the user's finger, and a mouthpiece may measure a user's expiratory pressure before and during a Valsalva maneuver. In some embodiments, the user may be directed (e.g., by a device connected to the sensor system presenting a graphical user interface) to perform a Valsalva maneuver and sustain a particular expiratory pressure for a period of time. Biometrics, such as PPG measurements and expiratory measurements may be obtained by the analysis system before and during the Valsalva maneuver. In some embodiments, the analysis system may execute a process, described in greater detail herein, to obtain a ratio between a baseline PPG RMS prior to the Valsalva maneuver, and minimum PPG RMS during the Valsalva maneuver. This ratio may be stored or outputted for aiding in a medical analysis (e.g., assessing cardiac filling pressure).

Certain embodiments of the disclosure will hereafter be described with reference to the accompanying drawings, wherein like reference numerals denote like elements. It should be understood, however, that the accompanying drawings illustrate only the various implementations described herein and are not meant to limit the scope of various technologies described herein. The drawings show and describe various embodiments of the current disclosure.

Embodiments of the disclosure may include a system, a method, and/or a computer program product at any possible technical detail level of integration. The computer program product may include a computer readable storage medium (or media) having computer readable program instructions thereon for causing a processor to carry out aspects of the present disclosure.

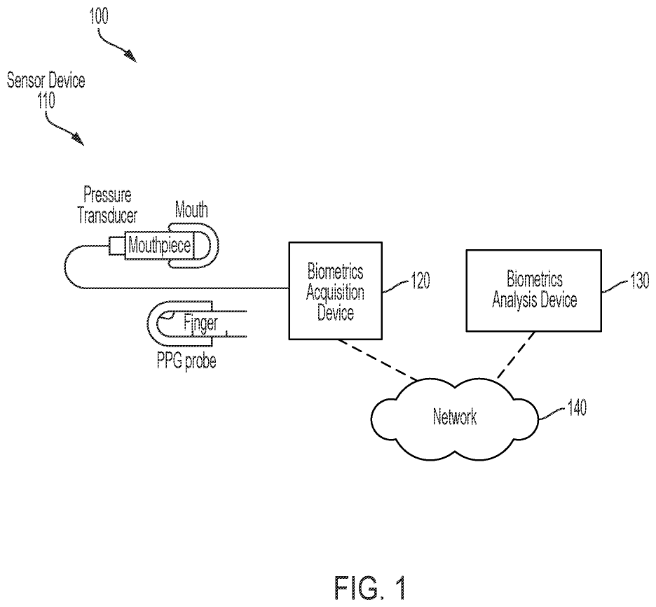

FIG. 1 illustrates an example overview and environment in accordance with aspects of the present disclosure. As shown in FIG. 1, environment 100 includes a sensor device, a biometrics acquisition device 120, a biometrics analysis device 130, and a network 140.

The sensor device 110 may include one or more biometrics sensors that obtain biometrics data from a patient. In some embodiments, the sensor device 110 may include a mouthpiece including a pressure transducer to measure expiratory pressure from the patient. The sensor device 110 may also include a PPG finger probe which may include a pulse oximeter which illuminates the skin and measures changes in light absorption. In some embodiments, sensor device 110 may obtain raw sensor data including expiratory pressure from the patient (e.g., via the mouth piece) and the light absorption (e.g., via the PPG finger probe) and provide the raw sensor data to the biometrics acquisition device 120.

The biometrics acquisition device 120 may include any a variety of computing device, such as a mobile or non-mobile computing device. The biometrics acquisition device 120 may host an application that obtains raw biometrics sensor data from the biometrics acquisition device 120. In some embodiments, the biometrics acquisition device 120 may present a graphical user interface (GUI) that guides the user (e.g., patient or study participant) to sustain a consistent expiratory pressure (e.g., of, 20 mmHg) for a duration of time (e.g., 10 seconds) in order to perform a Valsalva maneuver. In some embodiments, the biometrics acquisition device 120 may present a meter that identifies a current expiratory pressure to assist the user in maintaining a consistent expiratory pressure.

The biometrics analysis device 130 may include one or more computing devices that executes one or more processes for obtaining a ration between a minimum PPG RMS during the Valsalva maneuver, and a baseline PPG prior to the Valsalva maneuver. In some embodiments, the biometrics analysis device 130 may receive biometrics data (e.g., captured by the sensor device 110), model the biometrics data, determine a baseline RMS of a baseline PPG prior to the Valsalva maneuver, determine a minimum Valsalva RMS during the Valsalva maneuver, calculate a ratio between the minimum Valsalva RMS and the baseline RMS to obtain a minimum RMS ration, and store or output the minimum RMS ratio. As described herein, the minimum RMS ratio may be used as part of a medical analysis (e.g., to assess cardiac filling pressure or other type of medical analysis) without requiring waveform peaks and valleys to be determined, which may be difficult and inaccurate in patients with arrythmias. Additionally, or alternatively, the biometrics analysis device 130 may determining an RMS value at the end of the Valsalva maneuver, determine a ratio between the baseline RMS and the RMS value at the end of the Valsalva maneuver, and store or output this ratio as part of a medical analysis.

The network 140 may include network nodes and one or more wired and/or wireless networks. For example, the network 140 may include a cellular network (e.g., a second generation (2G) network, a third generation (3G) network, a fourth generation (4G) network, a fifth generation (5G) network, a long-term evolution (LTE) network, a global system for mobile (GSM) network, a code division multiple access (CDMA) network, an evolution-data optimized (EVDO) network, or the like), a public land mobile network (PLMN), and/or another network. Additionally, or alternatively, the network 140 may include a local area network (LAN), a wide area network (WAN), a metropolitan network (MAN), the Public Switched Telephone Network (PSTN), an ad hoc network, a managed Internet Protocol (IP) network, a virtual private network (VPN), an intranet, the Internet, a fiber optic-based network, and/or a combination of these or other types of networks. In embodiments, the network 140 may include copper transmission cables, optical transmission fibers, wireless transmission, routers, firewalls, switches, gateway computers and/or edge servers.

The quantity of devices and/or networks in the environment 100 is not limited to what is shown in FIG. 1. In practice, the environment 100 may include additional devices and/or networks; fewer devices and/or networks; different devices and/or networks; or differently arranged devices and/or networks than illustrated in FIG. 1. Also, in some implementations, one or more of the devices of the environment 100 may perform one or more functions described as being performed by another one or more of the devices of the environment 100. Devices of the environment 100 may interconnect via wired connections, wireless connections, or a combination of wired and wireless connections.

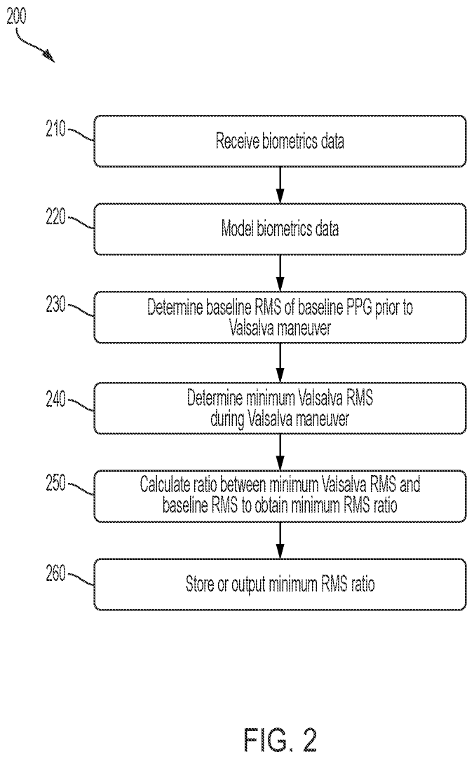

FIG. 2 illustrates an example flowchart of a process for determining a minimum RMS ratio that may be used in a medical analysis. For example, the process 200 of FIG. 2 may be performed as part of a procedure in which a user (e.g., patient, study participant, etc.) may perform a Valsalva maneuver as part of a medical analysis (e.g., medical examination, test, study, diagnosis, etc.). The blocks of FIG. 2 may be implemented in the environment of FIG. 2, for example, and are described using reference numbers of elements depicted in FIG. 2. As noted herein, the flowchart illustrates the architecture, functionality, and operation of possible implementations of systems, methods, and computer program products according to various embodiments of the present disclosure.

As shown in FIG. 2, The process 200 may include receiving biometrics data (block 210). For example, the biometrics analysis device 130 may receive biometrics data (e.g., for a user, such as a patient, study participant, etc.) from the sensor device 110 and/or the biometrics acquisition device 120. In some embodiments, the biometrics data may include expiratory pressure measurements and/or PPG measurements before and during a Valsalva maneuver. For example, the user may be directed (e.g., by a user interface of the biometrics acquisition device 120) to sustain a consistent expiratory pressure (e.g., of, 20 mmHg) for a duration of time (e.g., 10 seconds) in order to perform a Valsalva maneuver.

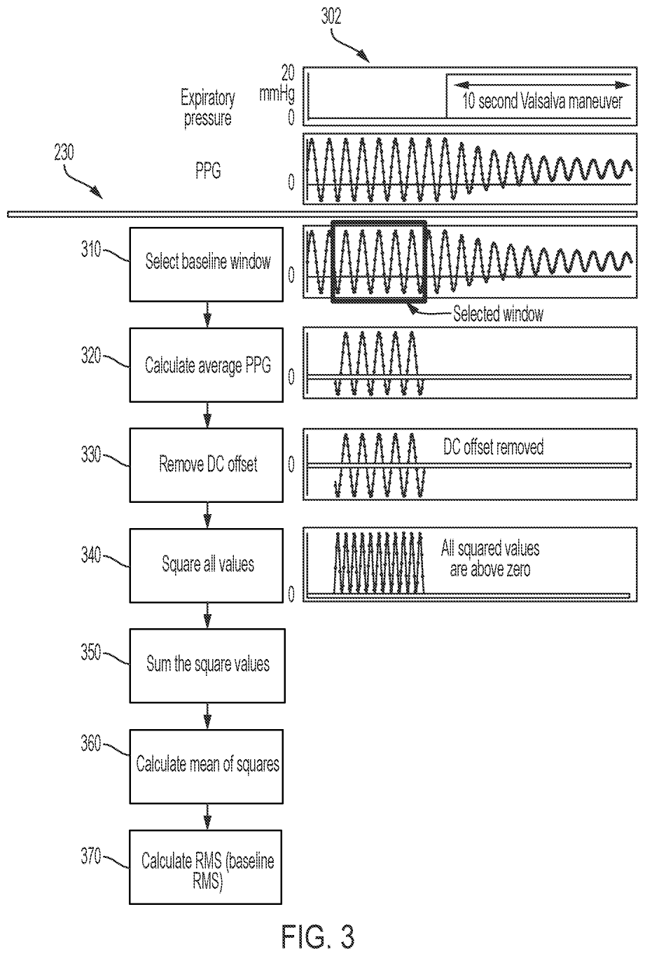

The process 200 also may include modeling the biometrics data (block 220). For example, the biometrics analysis device 130 may model the biometrics data (e.g., by plotting the biometrics data on a chart or graph). An example of the modeled biometrics data is shown in FIG. 3 (e.g., at graph 302). Referring to graph 302 in FIG. 3, the expiratory pressure and PPG may be graphed prior to and during a Valsalva maneuver. In the example shown, the Valsalva maneuver lasts 10 seconds in duration at an expiratory pressure of 20 mmHg. However, the techniques described herein are not limited to a particular Valsalva maneuver duration or expiratory pressure.

The process 200 further may include determining a baseline PPG prior to the Valsalva maneuver (block 230). For example, the biometrics analysis device 130 may determine a baseline PPG prior to the Valsalva maneuver. FIG. 3, illustrates an example sub-process for determining the baseline PPG. Referring to FIG. 3, determining the baseline PPG may include selecting a baseline PPG window (block 310). In some embodiments, the biometrics analysis device 130 may select a baseline PPG window prior to the Valsalva maneuver. The window may be of any duration, and in one example, may be approximately 3 seconds. As further shown in FIG. 3, determining the baseline PPG may further include calculating the average PPG at the selected window (block 320), and removing direct current (DC) offset (block 330) using any suitable DC offset removal technique. Determining the baseline PPG may further include squaring all values above zero (block 340), summing the squared values (block 350), and calculating the mean of all squared values (block 360). A t block 370, the baseline PPG is calculated from the mean of the squared values (obtained from block 360) using any suitable RMS calculation technique.

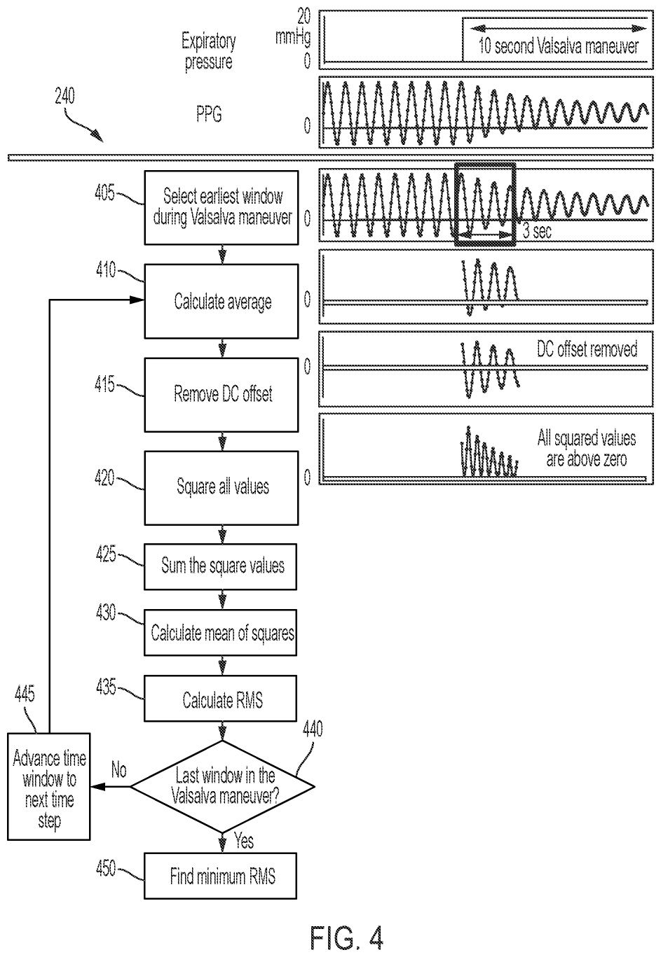

Referring back to FIG. 2, the process 200 also may include determining a minimum Valsalva RMS during the Valsalva maneuver (block 240). For example, the biometrics analysis device 130 may determine a minimum Valsalva RMS during the Valsalva maneuver. FIG. 4 illustrates an example sub-process for determining a minimum Valsalva RMS during the Valsalva maneuver. For example, referring to FIG. 4, determining the minimum Valsalva RMS during the Valsalva maneuver may include selecting the earliest time window during the Valsalva maneuver (block 405). In the example shown, the time window may be three seconds in duration, although a longer or shorter duration may be used. As further shown in FIG. 4, determining the minimum Valsalva RMS may further include calculating an average during the selected window (block 410), removing DC offset (block 415) (e.g., using any suitable DC offset removal technique), squaring values above zero (block 420), summing the squared values (block 425), calculating the mean of the squares (block 430), and calculating the RMS (block 435). The RMS for the selected window may be stored. At block 440, if the selected window is not the last window in the Valsalva maneuver (block 440-NO), the time window may be advanced to the next time step (block 445).

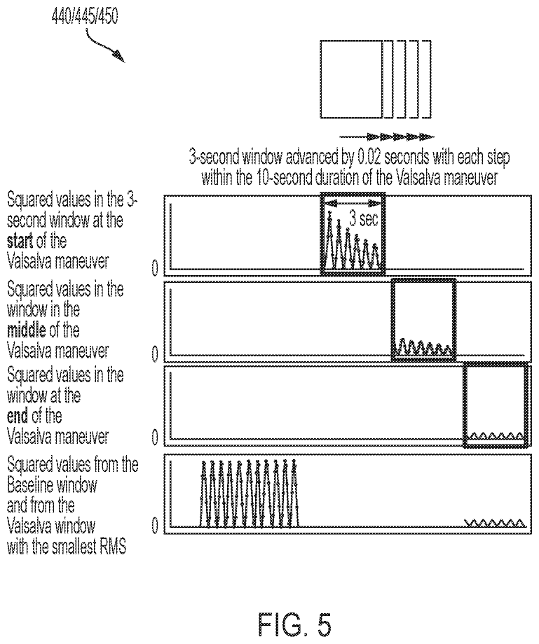

The process of FIG. 4 may be repeated in which a subsequent time window is selected within the Valsalva maneuver (e.g., a time window that is after the previously selected time window, such as 0.02 seconds after). In this way, the time window continues to advance the next time step (e.g., to the right until) the cumulative time windows have covered the entire Valsalva maneuver. For example, referring to FIG. 5, the time window (e.g., 3-second window) is advanced to the next time step (e.g., by 0.02 seconds, or other time step) within the duration of the Valsalva maneuver until all the entire Valsalva maneuver has been covered. As further shown in FIG. 5, the squared values at each time window is determined. The RMS at each time window during the Valsalva maneuver may be determined and stored (e.g., in accordance with the process of FIG. 4). All previously stored RMS values may be sorted. The smallest RMS during the Valsalva maneuver may be selected as shown in FIG. 5 and represented against the baseline RMS (determined at block 230 using the sub-process shown in FIG. 3). In brief, determining the minimum Valsalva RMS value may include iteratively calculating respective RMS values at iterative time windows during performance of the Valsalva maneuver, and identifying the minimum RMS value from these respective RMS values at different time windows.

In the example shown in FIG. 5, the minimum RMS value is at the end of the Valsalva maneuver, but in practice, this may not always be the case. As such, by determining the RMS values at different time windows and at different time steps, the minimum RMS value may be determined regardless of when the minimum RMS value occurs during the Valsalva maneuver. Additionally, or alternatively, the RMS value of the last segment during the Valsalva maneuver may be used. In this embodiment, the end of the Valsalva maneuver may be identified from the modeled biometrics data, a time window covering a period of time before the end of the Valsalva maneuver up until the end of the Valsalva maneuver may be selected, and the RMS value during this selected window may be determined.

Returning to FIG. 2, the process 200 further may include calculating a ratio between the minimum Valsalva RMS and the baseline RMS to obtain a minimum RMS ratio (block 250). For example, the biometrics analysis device 130 may calculate a ratio between the minimum Valsalva RMS (obtained at block 240) and the baseline RMS (obtained a block 230) to obtain the minimum RMS ratio.

The process 200 also may include storing or outputting the minimum RMS ratio (block 260). For example, the biometrics analysis device 130 may store or output the minimum RMS ratio. In some embodiments, the minimum RMS ratio may be presented on a display, output/stored to another device, included in a report or chart, etc.

As described herein, the minimum RMS ratio may be used for a medical analysis (e.g., a medical examination, test, study, etc.), such as an assessment of cardiac filling pressure (e.g., left heart filling pressure (LHFP) fluid status or state of congestion), or other type of medical analysis/examination without the need to analyze a PPG waveform, which may be unstable and inaccurate in patients with arrhythmias. In a testing environment, the minimum RMS ratio in patients with arrhythmias was compared to the RMS ratio in patients without arrhythmias with low deviation between the two types of patients. As such, using the minimum RMS ratio instead of PPG waveform analysis does not significantly affect the quality of medical analysis, and in fact, improves the quality of medical analysis for patients with arrythmias.

In an alternative embodiment, the minimum RMS ratio may be the ratio between the baseline RMS and the RMS value at the end of the Valsalva maneuver, rather than the minimum RMS during the Valsalva maneuver. This ratio between the baseline RMS and the RMS value at the end of the Valsalva maneuver may be stored and outputted for use as part of a medical examination, diagnosis, test, etc. without the need to analyze a PPG waveform.

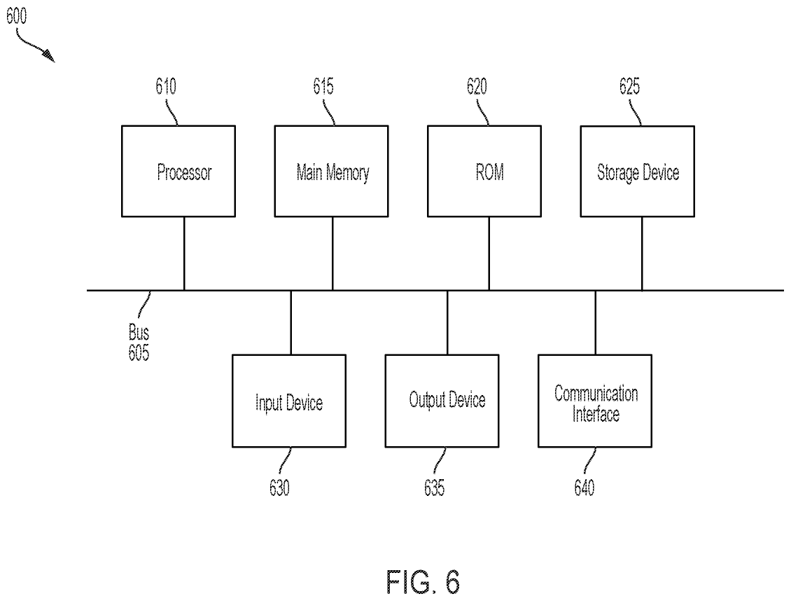

FIG. 6 illustrates example components of a device 600 that may be used within environment 100 of FIG. 1. Device 600 may correspond to the sensor device 110, the biometrics acquisition device 120, and/or the biometrics analysis device 130. Each of the sensor device 110, the biometrics acquisition device 120, and/or the biometrics analysis device 130 may include one or more devices 600 and/or one or more components of device 600.

As shown in FIG. 6, device 600 may include a bus 605, a processor 610, a main memory 615, a read only memory (ROM) 620, a storage device 625, an input device 630, an output device 635, and a communication interface 640.

Bus 605 may include a path that permits communication among the components of device 600. Processor 610 may include a processor, a microprocessor, an application specific integrated circuit (ASIC), a field programmable gate array (FPGA), or another type of processor that interprets and executes instructions. Main memory 615 may include a random access memory (RAM) or another type of dynamic storage device that stores information or instructions for execution by processor 610. ROM 620 may include a ROM device or another type of static storage device that stores static information or instructions for use by processor 610. Storage device 625 may include a magnetic storage medium, such as a hard disk drive, or a removable memory, such as a flash memory.

Input device 630 may include a component that permits an operator to input information to device 600, such as a control button, a keyboard, a keypad, or another type of input device. Output device 635 may include a component that outputs information to the operator, such as a light emitting diode (LED), a display, or another type of output device. Communication interface 640 may include any transceiver-like component that enables device 600 to communicate with other devices or networks. In some implementations, communication interface 640 may include a wireless interface, a wired interface, or a combination of a wireless interface and a wired interface. In embodiments, communication interface 640 may receive computer readable program instructions from a network and may forward the computer readable program instructions for storage in a computer readable storage medium (e.g., storage device 625).

Device 600 may perform certain operations, as described in detail below. Device 600 may perform these operations in response to processor 610 executing software instructions contained in a computer-readable medium, such as main memory 615. A computer-readable medium may be defined as a non-transitory memory device and is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. A memory device may include memory space within a single physical storage device or memory space spread across multiple physical storage devices.

The software instructions may be read into main memory 615 from another computer-readable medium, such as storage device 625, or from another device via communication interface 640. The software instructions contained in main memory 615 may direct processor 610 to perform processes that will be described in greater detail herein. Alternatively, hardwired circuitry may be used in place of or in combination with software instructions to implement processes described herein. Thus, implementations described herein are not limited to any specific combination of hardware circuitry and software.

In some implementations, device 600 may include additional components, fewer components, different components, or differently arranged components than are shown in FIG. 6.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments of the disclosure may include a system, a method, and/or a computer program product at any possible technical detail level of integration. The computer program product may include a computer readable storage medium (or media) having computer readable program instructions thereon for causing a processor to carry out or execute aspects and/or processes of the present disclosure.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on a user's computer, partly on the user's computer, as a stand-alone software package, partly on the user's computer and partly on a remote computer or entirely on the remote computer or server.

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In embodiments, a service provider could offer to perform the processes described herein. In this case, the service provider can create, maintain, deploy, support, etc., the computer infrastructure that performs the process steps of the disclosure for one or more customers. These customers may be, for example, any business that uses technology. In return, the service provider can receive payment from the customer(s) under a subscription and/or fee agreement and/or the service provider can receive payment from the sale of advertising content to one or more third parties.

The foregoing description provides illustration and description, but is not intended to be exhaustive or to limit the possible implementations to the precise form disclosed. Modifications and variations are possible in light of the above disclosure or may be acquired from practice of the implementations.

It will be apparent that different examples of the description provided above may be implemented in many different forms of software, firmware, and hardware in the implementations illustrated in the figures. The actual software code or specialized control hardware used to implement these examples is not limiting of the implementations. Thus, the operation and behavior of these examples were described without reference to the specific software code—it being understood that software and control hardware can be designed to implement these examples based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While the present disclosure has been disclosed with respect to a limited number of embodiments, those skilled in the art, having the benefit of this disclosure, will appreciate numerous modifications and variations there from. It is intended that the appended claims cover such modifications and variations as fall within the true spirit and scope of the disclosure.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

What is claimed is:

1. A computer-implemented method comprising:
receiving biometrics data for a user;
determining, based on the biometrics data, a baseline root-mean square (RMS) value prior to performance of a medical maneuver by the user;
determining a minimum RMS value of the biometrics data during the medical maneuver;
calculating a ratio between the minimum RMS value and the baseline RMS value to generate a minimum RMS ratio; and
storing or outputting the minimum RMS ratio.

2. The method of claim 1, wherein the biometrics data includes a photoplethysmogram (PPG) value and an expiratory pressure value.

3. The method of claim 1, wherein the medical maneuver is a Valsalva maneuver.

4. The method of claim 1, wherein the determining the baseline RMS value prior to performance of a medical maneuver by the user comprises:
selecting a baseline time window prior to the performance of the medical maneuver;
calculating an average photoplethysmogram (PPG) value during the selected baseline time window;
removing a direct current (DC) offset;
squaring values above zero after removing the DC offset;
summing the squared values;
calculating a mean of the squared values; and
calculating the baseline RMS value.

5. The method of claim 1, wherein the determining the minimum RMS value of the biometrics data during the medical maneuver comprises:
selecting a first time window during performance of the medical maneuver;
calculating an average photoplethysmogram (PPG) value during the selected baseline time window;
removing a direct current (DC) offset;
squaring values above zero after removing the DC offset;
summing the squared values;
calculating a mean of the squared values; and
calculating a first RMS value during the first time window;
advancing to a second time window;
calculating a second RMS value in the second time window; and
determining a minimum RMS value, of a plurality of RMS values calculated at respective plurality of time windows during performance of the medical maneuver.

6. The method of claim 1, wherein the determining the minimum RMS value of the biometrics data during the medical maneuver comprises:
iteratively calculating respective RMS values at iterative time windows during performance of the medical maneuver;
identifying the minimum RMS value from the respective RMS values.

7. The method of claim 1, wherein the minimum RMS ratio represents information pertaining to cardiac function.

8. A computer program product comprising a computer readable storage medium having program instructions embodied therewith, the program instructions executable by a computing device to cause the computing device to perform operations comprising:
receiving biometrics data for a user;
determining, based on the biometrics data, a baseline root-mean square (RMS) value prior to performance of a medical maneuver by the user;
determining a minimum RMS value of the biometrics data during the medical maneuver;
calculating a ratio between the minimum RMS value and the baseline RMS value to generate a minimum RMS ratio; and
storing or outputting the minimum RMS ratio.

9. The computer program product of claim 8, wherein the biometrics data includes a photoplethysmogram (PPG) value and an expiratory pressure value.

10. The computer program product of claim 8, wherein the medical maneuver is a Valsalva maneuver.

11. The computer program product of claim 8, wherein the operations for determining the baseline RMS value prior to performance of a medical maneuver by the user further comprises:
selecting a baseline time window prior to the performance of the medical maneuver;
calculating an average photoplethysmogram (PPG) value during the selected baseline time window;
removing a direct current (DC) offset;
squaring values above zero after removing the DC offset;
summing the squared values;
calculating a mean of the squared values; and
calculating the baseline RMS value.

12. The computer program product of claim 8, wherein the operations for determining the minimum RMS value of the biometrics data during the medical maneuver further comprises:
selecting a first time window during performance of the medical maneuver;
calculating an average photoplethysmogram (PPG) value during the selected baseline time window;
removing a direct current (DC) offset;
squaring values above zero after removing the DC offset;
summing the squared values;
calculating a mean of the squared values; and
calculating a first RMS value during the first time window;
advancing to a second time window;
calculating a second RMS value in the second time window; and
determining a minimum RMS value, of a plurality of RMS values calculated at respective plurality of time windows during performance of the medical maneuver.

13. The computer program product of claim 8, wherein the determining the minimum RMS value of the biometrics data during the medical maneuver comprises:
iteratively calculating respective RMS values at iterative time windows during performance of the medical maneuver;
identifying the minimum RMS value from the respective RMS values.

14. The computer program product of claim 8, wherein the minimum RMS ratio represents information pertaining to cardiac function.

15. A system comprising:
a processor, a computer readable memory, a non-transitory computer readable storage medium associated with a computing device, and program instructions executable by the computing device to cause the computing device to perform operations comprising:
receiving biometrics data for a user;
determining, based on the biometrics data, a baseline root-mean square (RMS) value prior to performance of a medical maneuver by the user;
determining an RMS value of the biometrics data at the end of the medical maneuver;
calculating a ratio between the the RMS value at the end of the medical maneuver and the baseline RMS value to generate an RMS ratio; and
storing or outputting the RMS ratio.

16. The system of claim 15, wherein the biometrics data includes a photoplethysmogram (PPG) value and an expiratory pressure value.

17. The system of claim 15, wherein the medical maneuver is a Valsalva maneuver.

18. The system of claim 15, wherein the operations for determining the baseline RMS value prior to performance of a medical maneuver by the user further comprises:
selecting a baseline time window prior to the performance of the medical maneuver;
calculating an average photoplethysmogram (PPG) value during the selected baseline time window;
removing a direct current (DC) offset;
squaring values above zero after removing the DC offset;
summing the squared values;
calculating a mean of the squared values; and
calculating the baseline RMS value.

19. The system of claim 15, wherein the operations for determining the RMS value of the biometrics data at the end of the medical maneuver comprise:
modeling the biometrics data;
identifying the end of the medical maneuver prior based on the modeling the biometrics data;
selecting a time window covering a period of time before the end of the medical maneuver up until the end of the medical maneuver; and
and calculating the RMS value within the selected window.

20. The system of claim 15, wherein the RMS ratio represents information pertaining to cardiac function.
